(19) United States
(12) Patent Application Publication
(10) Pub. No.: US 2009/0069795 A1
(43) Pub. Date: Mar. 12, 2009

(54) APPARATUS AND METHOD FOR SELECTIVE TREATMENT OF TISSUE

(76) Inventor: Robert S. Anderson, Livermore, CA (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 11/852,990

(22) Filed: Sep. 10, 2007

(51) Int. Cl. A61B 18/04

(52) U.S. Cl. 606/9; 607/88; 607/96

(57) ABSTRACT

Methods, systems, and devices to selectively treat a target tissue located in a region of skin. The region of skin undergoes a series of energy applications to selectively treat a target tissue. The region of skin may also be cooled to reduce damage to surrounding tissue.

### BACKGROUND OF THE INVENTION

1). Field of the Invention

The present invention relates to methods and devices useful in modification, treatment, destruction, and/or removal of tissue.

2). Discussion of Related Art

Any object heated to a temperature higher than the temperature of its surrounding environment will eventually cool to the temperature of its surrounding environment. The time it takes for an object to return the preheated temperature is called the thermal relaxation time. This time is a function of the physical parameters of the object such as its dimensions and composition. It is also a function of the means by which heat is transferred from the object to its environment. If two objects with different thermal relaxation times are heated simultaneously to the same temperature, and then allowed to cool, after a certain amount of time, one object will be at a higher temperature than the other. The object with the longer thermal relaxation time will be at the higher temperature.

If two objects with different thermal relaxation times are heated simultaneously to either the same temperature or different temperatures and then allowed to cool, after a certain amount of time, one object may close to the temperature of its environment while the object with the longer thermal relaxation time may be at an elevated temperature relative to its environment. If the two objects are subjected again to a heat source, the object with the longer thermal relaxation time will go to an even higher temperature than it reached after its first increase in temperature. The object with the shorter thermal relaxation time, however, will rise to a temperature close to the temperature it rose to on the first increase in temperature.

Unwanted biological tissue may be heated to a destructive temperature in order to destroy the tissue. Examples of biological tissues that may be heated to a destructive temperature include vascular lesions, hair follicles, sebaceous glands, pigmented lesions of collagen, and adipose tissue. Often unwanted biological target tissue is situated directly in the vicinity of wanted biological tissue, for example an undesired hair located in the epidermis. Previous therapies of removing unwanted biological tissue by an application of energy (e.g. coherent and non-coherent light, RF, heat) often are also destructive to surrounding tissue. Previous therapies are sometimes effective at tissue removal, for example laser hair removal, but also cause unnecessary burning, pain, discomfort, extended healing time, and inconsistent results.

### SUMMARY OF THE DESCRIPTION

The description provides a method to destroy a target tissue while leaving surrounding tissue unharmed, the method including heating a target tissue and surrounding tissue to a temperature below what is required to destroy the target tissue and surrounding tissue, wherein the target tissue has a thermal relaxation time which is greater than a thermal relaxation time of the surrounding tissue, allowing the target tissue and surrounding tissue to cool for a time interval, wherein the target tissue does not cool as much as the surrounding tissue cools, and reheating the target tissue and surrounding tissue wherein the target tissue is destroyed and the surrounding tissue is not destroyed. The method may also include determining that the target tissue will have a greater thermal relaxation time than the surrounding tissue before beginning the treatment in order to assure that the treatment will affect the target tissue and not the other tissue.

The description also includes a system to destroy a target tissue while leaving surrounding tissue unharmed, the system including means for heating a target tissue and surrounding tissue to a temperature below what is required to destroy the target tissue and surrounding tissue, wherein the target tissue has a thermal relaxation time which is greater than a thermal relaxation time of the surrounding tissue, means for allowing the target tissue and surrounding tissue to cool for a time interval, wherein the target tissue does not cool as much as the surrounding tissue cools, and means for reheating the target tissue and surrounding tissue wherein the target tissue is destroyed and the surrounding tissue is not destroyed.

The description also includes a method to destroy a target tissue while leaving surrounding tissue unharmed, the method including applying a plurality of energy applications to a target tissue and surrounding tissue, wherein between each energy application the target tissue and surrounding tissue are allowed to cool for a time interval such during the time interval more heat is retained by the target tissue than the surrounding tissue, and wherein the target tissue eventually retains enough heat to destroy the target tissue while concurrently the surrounding tissue does not retain enough heat to be destroyed.

The description also includes a system to destroy a target tissue while leaving surrounding tissue unharmed, the system including means for applying a plurality of energy applications to a target tissue and surrounding tissue, wherein between each energy application the target tissue and surrounding tissue are allowed to cool for a time interval such that during the time interval more heat is retained by the target tissue than the surrounding tissue, and wherein the target tissue eventually retains enough heat to destroy the target tissue while concurrently the surrounding tissue does not retain enough heat to be destroyed.

### DESCRIPTION OF THE DRAWINGS

The present invention is illustrated by way of example and not limitation in the figures of the accompanying drawings in which like references indicate similar elements.

FIG. 1 is a cross-sectional view of human skin tissue.

FIG. 2 is a cross-sectional view of a device for treating human tissue.

FIG. 3 is graph illustrating heating and cooling of target tissue and surrounding tissue.

FIG. 4 is a flow chart illustrating a method to destroy target tissue while not harming surrounding tissue.

FIG. 5 is a diagram of a system to destroy target tissue while not harming surrounding tissue.

### DETAILED DESCRIPTION OF THE INVENTION

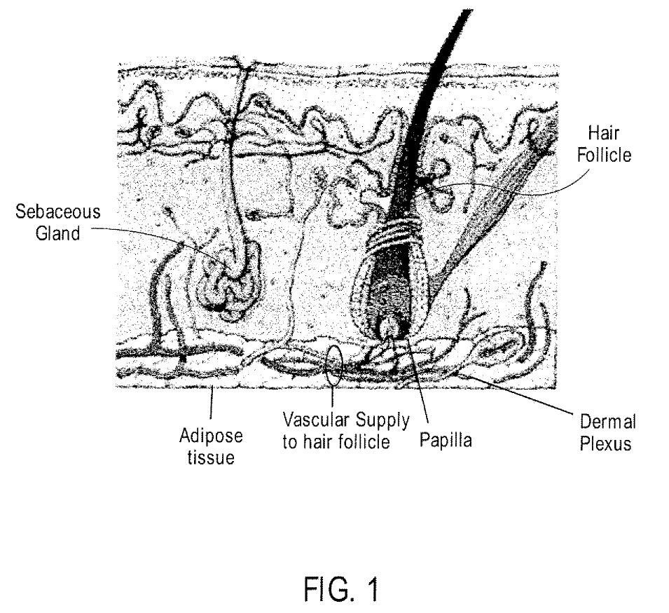

FIG. 1 is a cross section showing the various layers of the skin, the epidermis and dermis, and potential targets for treatment, which include tissues such as hair follicles, hair papilla, adipose tissue, sebaceous glands, and pigmented lesions of collagen. As shown, the tissue includes a hair follicle. At the base of the hair follicle is a large bulb like structure (hair bulb) which covers a knob like vascular indentation called the papilla, the hair bulb and the papilla for the purposes of this disclosure will be named the papilla. The papilla is the largest part of the hair follicle, and interfaces with the blood stream to carry nutrients to the hair follicle for growth of hair. Thus in order to stop the growth of new hair, any treatment must destroy the papilla in order to prevent the growth of new hair.

When energy first impacts the skin, it encounters the epidermis, the outer most layer of the skin. The papilla resides significantly below the outer layer of the epidermis and receives less externally applied energy than the epidermis, accordingly the amount of energy required to destroy the papilla by transferring heat from a single energy application applied externally to the epidermis would also destructive to the surrounding tissue (epidermis) of the papilla. Previous therapies of laser hair removal have been inconsistent in permanent hair removal because on some patients the papilla is located too deep to heat to destruction without seriously damaging the epidermis. Previous therapies of laser removal tend to target the hair follicle region well above the papilla which is not difficult since it is close to the surface, but are also ineffective because those therapies do not destroy the papilla. One embodiment of the current invention proposes to apply cyclical energy applications in order to provide a gradual build up of destructive heat in the papilla, while simultaneously allowing surrounding tissue to retain heat which is respectively non-destructive.

The surrounding tissue requires a shorter thermal relaxation time than the papilla in order to prevent damage to the surrounding tissue. The thermal relaxation time of the tissue surrounding the papilla may be reduced by cooling the epidermis. Cooling the epidermis will not significantly affect the thermal relaxation time of the papilla, because the papilla is located well beneath the outer layer of the epidermis. The papilla is also of significant mass and has different material properties than the surrounding tissue, both of which contribute to a longer thermal relaxation time. Thus energy applied cyclically in conjunction with cooling of the epidermis will result in the eventual destruction of the papilla without harm to surrounding tissue, because heat will not sufficiently be dissipated from the papilla. It is important to nota that the destruction of the papilla is merely given as an example of a target tissue, which this disclosure is not limited to. Other target tissues with a thermal relaxation time longer than surrounding tissue and are applicable to embodiments disclosed herein include vascular lesions, adipose tissue, pigmented lesions of collagen, and the sebaceous gland.

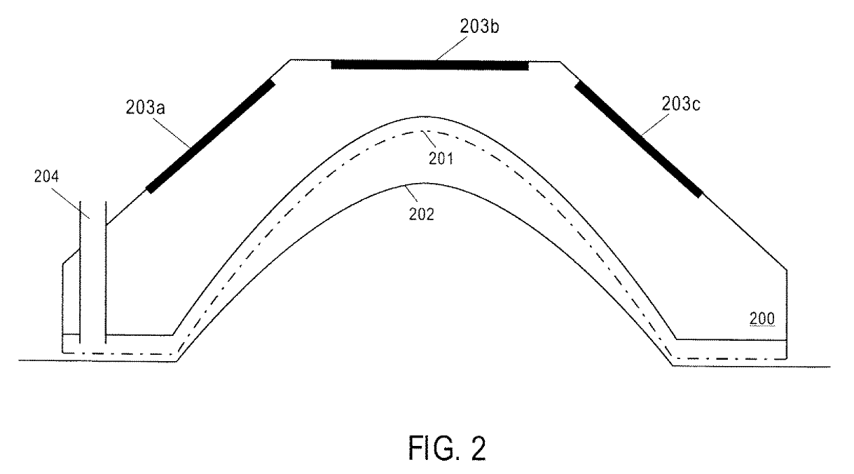

FIG. 2 shows a device for destroying target tissue without harming the surrounding tissue. The device is similar to devices shown in commonly assigned and invented U.S. patent application Ser. No. 11/024,340, published as US 2005-0251118 A1, and U.S. patent application Ser. No. 11/123,599, published as US 2006-0189964 A1, the both of which are hereby incorporated by reference in their entirety.

FIG. 2 shows, in cross-sectional view, a device 300 having multiple light sources 203a, 203b, and 203c, and a pressure conduit 204. The light sources are contained within a housing and/or body which also includes a cover (which is transparent in the case of light sources) and which separates the light sources from any vacuum generated between the skin and the device). The cover is disposed between the membrane 201 and the light sources 203a-203c. A handle which is coupled to the body may also be included so that a user of the device can easily hold and move the device over a patient's skin and/or other biological external tissue.

A recess and/or void exists between the membrane 201, which faces the biological external tissue 202, and the biological external tissue 202 shown in FIG. 2. Pressure conduit 204 generates a negative vacuum through membrane 201 to bring the biological external tissue 202 into the recess and toward the membrane 201. Membrane 201 can be used to collect dead skin, according to one embodiment of the invention. The membrane 201 is coupled to the conduit 204 to receive the suction from a vacuum pump (not shown) which is coupled to the conduit 204. Light sources 203a, 203b and 203c in FIG. 2 are connected to an energy source that is not shown on the figure, according to one embodiment of the invention. This energy source is not exposed to any pressure through the pressure conduit 204, according to one embodiment of the invention. These light sources are shielded from any negative (or positive) pressure by the cover which is optically transparent in the case where the energy sources provide visible light. It will be appreciated that the light sources may alternatively be other types of energy sources (e.g., microwave radio frequency energy) which may not require an optically transparent cover.

The energy applied to biological external tissue 202 through device 200 is transferred through light sources 203a, 203b and 203c. The light sources 203a, 203b, and 203c may include, for example, light emitting diode (LED) lasers of different wavelengths, thus providing different energy sources, due to the different wavelengths, in the body of the device. Each light source (e.g., source 203a and/or 203b and/or 203c) may be a panel of multiple LED lasers which may be the same type of LED (to produce the same wavelength) and/or may be a panel of multiple LED lasers which may be a different type of LED (to produce different wavelengths). The three panels shown in FIG. 2 (light sources 203a, 203b, and 203c) are arranged within the body of device 200 to provide a spatially uniform lighting at the target so that the intensity of light, at any point over an area which includes the target, is substantially the same. It can be seen from FIG. 2 that the panels (e.g., light source 203a) transmit light directly to the target without any intervening optical fibers and/or waveguides. The energy applied to biological external tissue 202 through device 200 may be applied repetitively until the desired effect on the tissue 202 is achieved.

This energy for device 200 can be incoherent light, coherent light, and/or alternatively non-visible light and/or electromagnetic radiation in the range of a radio frequency spectrum, and/or ultrasound, according to various embodiments of the invention. The energy source for the device 200 may be a flash lamp, arc lamp, high frequency electrical energy, RF energy, an LED and/or a Direct Current electrical energy. The device 200 invention may also include multiple combinations of different energies which are provided by energy sources in the body of the device 200. The device 200 may also be connected to a pressure source in the device 200 for providing power to the device 200 and generating pressure through a pressure conduit 204 in one embodiment of the invention. In another embodiment of the invention, the device 200 may be a handheld device that is connected to the pressure source (through a cable element), where the pressure Source and power source is separate from the handheld device. In addition, a controller on and/or near device 200 may control the strength of the energy applied through the light source 203a, 203b and/or 203c. According to one embodiment of the invention, there are three light sources however; any number of light sources is contemplated by the present invention. In one embodiment of the invention, a tapered outer wall on the periphery of device 200 prevents the biological external tissue 202 that is outside the device 200 from stretching.

A material, (e.g., a liquid such as water and/or ethyl alcohol, and/or other solid, liquid and/or gas substance having desired properties), may be applied to cool the biological external tissue 202. In one embodiment, the material is applied through a conduit on the device 200. Materials that provide little evaporative cooling at atmospheric pressure may provide significant evaporative cooling at pressures less than one atmosphere. Water, for example, provides little evaporative cooling at atmospheric pressure, but “boils” at 60° C. in one third of an atmosphere and can provide significant evaporative cooling at one third of an atmosphere. There are other materials, substances, and liquids that could be used effectively for the material. An important criterion is that the material, at a desired temperature, has a vapor pressure equal to or higher than the pressure inside the device 200 during treatment, (e.g., application of energy). Many alcohols meet this criterion. Ethyl alcohol has a vapor pressure of −15 PSI at 57° C. Its heat of vaporization is 854 Joules per gram which is less than water's 2450 Joules per gram. Nevertheless, ethyl alcohol may also provide elevated cooling at 55° C. as it carries off excess heat by vaporizing. In one embodiment, the material is applied prior to a treatment. In another embodiment, the material is applied as a spray, wiped out using a sponge and/or other object and/or in any other suitable manner. Alternatively additional methods of cooling may be employed either in lieu of or combined with evaporative cooling. Examples include applying a cooling gas or liquid at atmospheric pressure through a conduit (not shown) in the device 200, or by applying a cold, optically transparent element (not shown) on the biological external tissue 202, prior to, during and after treatment.

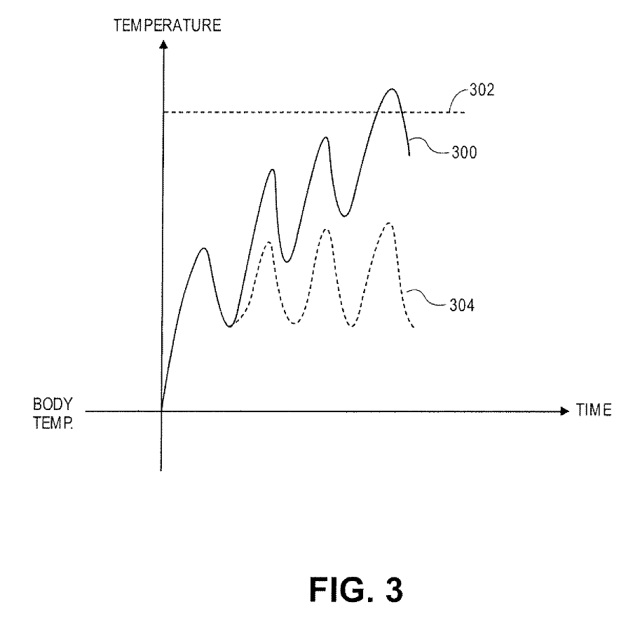

FIG. 3 graphically depicts the heating and cooling cycle of target tissue (e.g. papilla) and the surrounding tissue (e.g. epidermis) when undergoing treatment for example by a device similar to what is shown in FIG. 2. Curve 300 slows a heating and cooling curve of a target tissue, which may include a hair papilla, vascular lesions adipose tissue, pigmented lesions of collagen, and the sebaceous gland. The curve 300 has several peaks, which each represent cyclical (e.g. a plurality) applications of energy. In the curve 300 shown, the target tissue is subjected to four heating cycles, where upon each cycle the target tissue retains energy in the form of heat. After each heating cycle the target tissue retains a significant amount of heat compared to what is dissipated. The temperature at which the target tissue is destroyed is show by line 302. More or less heating cycles may be required to destroy the target tissue than what is illustrated, generally at least two cycles will be required.

Curve 304 shows a heating and cooling curve of tissue surrounding the target tissue. In the curve 304 shown, the tissue is subjected to four heating cycles, where upon each cycle the tissue may retain some heat energy. After each heating cycle the tissue retains an insignificant amount of heat compared to what is dissipated, which is not the case for the target tissue, as the tissue has a faster thermal relaxation time than the target tissue. The thermal relaxation time of the tissue may be decreased by evaporative cooling. The temperature at which the tissue is destroyed is shown by line 302, which is not attained during the heating cycles. A sufficient amount of time is required to allow the tissue to cool while allowing the target tissue to retain heat, for example a range between 50 and 1000 milliseconds may be acceptable.

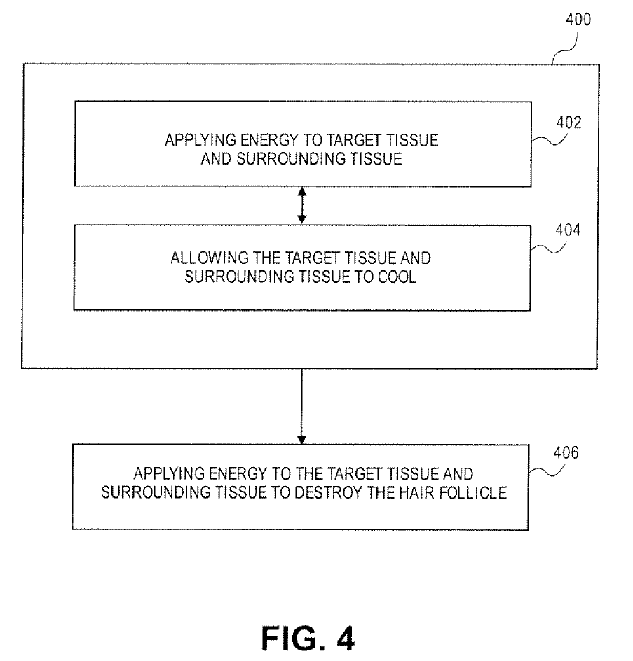

FIG. 4 shows a flowchart for a method to destroy a target tissue while not harming the surrounding tissue. Module 400 includes modules 402 and 404 which cycle between each other. In module 402 both the target tissue and surrounding tissue are subjected to energy which causes respective rises in temperature. The energy may be applied through the device 200 shown in FIG. 2. During the application of energy the surrounding tissue is heated, but not to a destructive temperature.

In module 404 the target tissue and surrounding tissue are allowed to cool, after a suitable amount of time the target tissue and surrounding tissue cool, but the surrounding tissue is cooled near to a pre-treatment temperature, while the target tissue retains a significant amount of heat. Energy is not applied in module 404. However cooling the surrounding tissue may occur in module 402 as well. Alternatively, cooling may occur only once initially before module 402 or module 404, or alternatively cooling may take place at every cycle, or a predetermined amount of times, for example after or during every third application of energy.

Modules 402 and 404 will cycle until the temperature of the target tissue becomes self destructive, at which the method proceeds to module 406 at which the last application of energy occurs to destroy the target tissue. In order for the surrounding tissue to remain unharmed over a number of cycles, the thermal relaxation time of the surrounding tissue needs to be shorter than the thermal relaxation time of the target tissue. Evaporative cooling, as described above, and other cooling methods may be employed to shorten the thermal relaxation time of the surrounding tissue.

Additionally the tissue may also be stretched during treatment, for example through negative pressure as applied through device 200. Stretching the tissue helps expose the target tissue to a greater degree of energy application by bringing it closer to the surface, and thus may require less cycles of energy application.

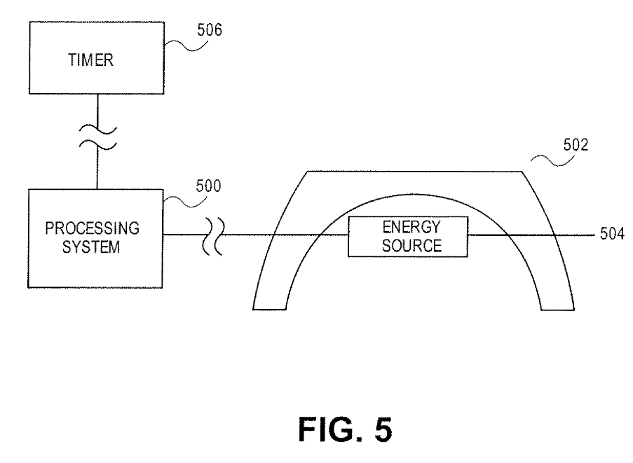

FIG. 5 shows a system implementing hardware for destroying target tissue without harming the surrounding tissue. The system includes a processing system 500 which applies energy to a device 502. The device 502 may be as shown in FIG. 2. The device 502 includes an energy source 504 for exposing energy to tissue. The processing system 500 is coupled to a timer 506. The timer 506 may be clock mechanism (e.g. a real time clock) or a software based timer or a crystal or other component for providing a representation of time or increments of time. The processing system 500 may include at least one microcontroller with instructions in the form of software for applying energy to the energy source 504 depending on a type of target. The software may not allow treatment of tissue unless the target tissue is determined to have a greater thermal relaxation time than the surrounding tissue. A user of the processing system 500 may be required to enter a type of target tissue (e.g. hair, adipose tissue) and surrounding tissue type before the processing system. Alternatively the processing system 500 may be preconfigured for one target tissue and may not include software. Once the tissue target is determined the processing system 500 may configure a treatment application for the destruction of the target tissue without harming surrounding tissue, where the processing system 500 uses the timer 506 to apply energy to the energy source 504 at predetermined intervals. These intervals are configured to allow the surrounding tissue to cool enough that it will not be destroyed (or otherwise not treated) by the repeated application of energy while the target tissue will be destroyed (or otherwise treated). The processing system 500 is particularly advantageous over past systems which did not provide for treatment of a target tissue without harming, surrounding tissue.

While certain exemplary embodiments have been described and shown in the accompanying drawings, it is to be understood that such embodiments are merely illustrative and not restrictive of the current invention, and that this invention is not restricted to the specific constructions and arrangements shown and described since modifications may occur to those ordinarily skilled in the art.

What is claimed:

1. A method to destroy a target tissue while leaving surrounding tissue unharmed, the method comprising:
heating a target tissue and surrounding tissue to a temperature below what is required to destroy the target tissue and surrounding tissue, wherein the target tissue has a thermal relaxation time which is greater than a thermal relaxation time of the surrounding tissue;
allowing the target tissue and surrounding tissue to cool for a time interval, wherein the target tissue does not cool as much as the surrounding tissue cools; and
reheating the target tissue and surrounding tissue wherein the target tissue is destroyed and the surrounding tissue is not destroyed.

2. The method as in claim 1 wherein heating and reheating are performed by a light source.

3. The method as in claim 1 additionally comprising cooling the surrounding tissue.

4. The method as in claim 3 wherein cooling comprises applying a liquid on the surrounding tissue and lowering air pressure around the surrounding tissue to cause the liquid to evaporate.

5. The method as in claim 1 wherein the time interval is between 50 and 1000 milliseconds.

6. The method as in claim 1 additionally comprising stretching the surrounding tissue.

7. The method as in claim 6 wherein stretching is performed by applying a positive and negative pressure to the surrounding tissue.

8. The method as in claim 1 wherein the target tissue is one of hair papilla, vascular lesions, adipose tissue, pigmented lesions of collagen, or a sebaceous gland.

9. A system to destroy a target tissue while leaving surrounding tissue unharmed, the system comprising:
a device for providing energy applications to a target tissue;
a processing system coupled to the device and wherein the processing system is configured to regulate energy applications of the device depending on a thermal relaxation time of the target tissue; and
a timer coupled to the processing system, the timer being used to allow the energy applications to be timed based on the thermal relaxation time.

10. The system as in claim 9 wherein the energy applications are performed by a light source.

11. The system as in claim 9 wherein the device provides cooling for non-target tissue surrounding tissue.

12. The system as in claim 11 wherein cooling comprises applying a liquid on the surrounding tissue and lowering air pressure around the surrounding tissue to cause the liquid to evaporate.

13. The system as in claim 9 wherein the processing system regulates energy applications at a time interval between 50 and 1000 milliseconds.

14. The system as in claim 9 wherein the device stretches the target tissue during at least one of before, during, and after the energy applications.

15. The system as in claim 14 wherein the device stretches by applying a positive and negative pressure to the target tissue.

16. The system as in claim 9 wherein the target tissue is one of hair papilla, vascular lesions, adipose tissue, pigmented lesions of collagen, or a sebaceous gland.

17. A method to destroy a target tissue while leaving surrounding tissue unharmed, the method comprising:
applying a plurality of energy applications to a target tissue and surrounding tissue, wherein between each energy application the target tissue and surrounding tissue are allowed to cool for a time interval such during the time interval more heat is retained by the target tissue than the surrounding tissue, and wherein the target tissue eventually retains enough heat to destroy the target tissue while concurrently the surrounding tissue does not retain enough heat to be destroyed.

18. The method as in claim 17 wherein the energy applications are performed through light energy.

19. The method as in claim 17 additionally comprising cooling the surrounding tissue.

20. The method as in claim 19 wherein evaporative cooling comprises applying a liquid on the surrounding tissue and lowering air pressure around the surrounding tissue to cause the liquid to evaporate.

21. The method as in claim 17 wherein the time interval is between 50 and 1000 milliseconds.

22. The method as in claim 17 additionally comprising stretching the surrounding tissue.

23. The method as in claim 17 wherein stretching is performed by applying a positive and negative pressure to the surrounding tissue.

24. The method as in claim 17 wherein the target tissue is one of hair papilla, vascular lesions, adipose tissue, pigmented lesions of collagen, or a sebaceous gland.

25. A system to destroy a target tissue while leaving surrounding tissue unharmed, the system comprising:
a processing system including a microprocessor having software for applying a plurality of energy applications to a target tissue and surrounding tissue wherein between each energy application the target tissue and surrounding tissue are allowed to cool for a time interval such during the time interval more heat is retained by the target tissue than the surrounding tissue, and wherein the target tissue eventually retains enough heat to destroy the target tissue while concurrently the surrounding tissue does not retain enough heat to be destroyed; and
a timer coupled to the processing system.
